(19) United States
(12) Patent Application Publication
(10) Pub. No.: US 2016/0022509 A1
(43) Pub. Date: Jan. 28, 2016

(54) ABSORBENT ARTICLE FOR ACCOMMODATING A CATHETER AND TUBING

(71) Applicant: REDDYCARE, LLC, Cincinnati, OH (US)

(72) Inventor: Usha Reddy, Cincinnati, OH (US)

(73) Assignee: REDDYCARE, LLC, Cincinnati, OH (US)

(21) Appl. No.: 14/774,811

(22) PCT Filed: Mar. 13, 2014
(86) PCT No.: PCT/US14/25643

§ 371 Date: Sep. 11, 2015

Related U.S. Application Data
(60) Provisional application No. 61/779,291, filed on Mar. 13, 2013.

(51) Int. Cl. A61F 13/49; A61M 25/02; A61F 13/47

(52) U.S. Cl. CPC A61F 13/49; A61F 13/47; A61M 25/02; A61F 2013/4506

(57) ABSTRACT

An absorbent article for use with an indwelling catheter is provided, the absorbent article including: a top sheet and a back sheet; an aperture for receiving a catheter, wherein the aperture traverses the top sheet and the back sheet, and wherein the aperture is proximate to an exit site of the catheter from an individual; a reclosable opening that traverses the top sheet and the back sheet, wherein the reclosable opening extends from the aperture to an edge of the absorbent article; and a fastener for closing the re-closable opening. An insert for accommodating an indwelling catheter and an absorbent article for use with a medical device are also provided.

### TECHNICAL FIELD

The present application is directed to absorbent articles and inserts designed to accommodate catheters and associated tubing.

### BACKGROUND ART

Surgical procedures frequently require the placement and maintenance of an indwelling catheter. Particularly in the case of infants and individuals with bowel incontinence, the use of diapers or other absorbent articles presents a unique challenge in dealing with the tubing associated with pelvic catheter use. For example, infant males who undergo surgical correction for hypospadias repair are often discharged from the hospital with an indwelling urinary catheter that must remain in place for one to four weeks while surgical wounds heal. During that time, the caregiver must care for the surgical site and manage the tubing extending from the indwelling catheter, while also regularly changing the child's diaper and taking care to keep the surgical site clean and free of body exudates, such as fecal waste.

Parents are often instructed to accommodate external tubing extending from the catheter by cutting a hole in the front of a conventional disposable diaper, approximately in the area where the tubing extends from the body, through which the catheter tubing will pass. A second disposable diaper is then applied over the first diaper to collect any leaked waste, including fecal waste. This method is fraught with problems. First, most disposable diapers have an internal layer containing superabsorbent polymers for absorbing liquid waste. When a diaper is cut open, these polymer crystals leak out of the diaper and directly onto the wearer's skin, clothing, etc. Second, the method requires two diapers at each diaper change, which can be costly and a waste of resources. Third, wearing two diapers is bulky and can be uncomfortable for the child. Fourth, the method is inconvenient for the caregiver and difficult to manage when the catheter is already in place and connected to an exterior bag, since the caregiver must somehow feed the tubing through the hole in the first diaper. Either the caregiver must cut a large hole to accommodate the bag as well, or else the caregiver must disconnect the tubing from the bag, pass the tubing through the hole, and then reconnect the bag, neither option being ideal.

U.S. Pat. No. 6,014,777, issued to Gupton, describes a shorts-type undergarment with two vertical openings in the front of the shorts for maintenance of a catheter. However, Gupton is designed for use by an otherwise continent individual and fails to teach or suggest an absorbent article that would absorb and contain body exudates while still permitting access to a catheter and its associated tubing. The design of the Gupton garment, including the front openings, would permit ready leakage of fecal waste and urine.

A need exists for a convenient absorbent article that is designed to accommodate the tubing associated with indwelling catheters, which provides access to permit a caregiver to easily change an individual's diaper while the catheter is in place and which prevents leakage of exudates from the interior of the absorbent article during catheter usage.

### SUMMARY OF INVENTION

Disclosed herein are absorbent articles designed to accommodate indwelling catheters. In one embodiment, an absorbent article for use with an indwelling catheter is provided, the absorbent article comprising: a top sheet and a back sheet; an aperture for receiving a catheter, wherein the aperture traverses the top sheet and the back sheet, and wherein the aperture is proximate to an exit site of the catheter from an individual; a reclosable opening that traverses the top sheet and the back sheet, wherein the reclosable opening comprises a first side and a second side and wherein the reclosable opening extends from the aperture to an edge of the absorbent article; a fastener for closing the reclosable opening; and an absorbent core disposed between the top sheet and the back sheet, wherein the aperture and the reclosable opening each traverse the top sheet, the absorbent core, and the back sheet.

In another embodiment, an insert is provided, the insert comprising a non-flexible cup comprising an aperture for receiving a catheter, wherein the aperture is proximate to an exit site of the catheter from an individual; and a reclosable opening that extends from the aperture to an edge of the non-flexible cup, wherein the relosable opening comprises a first side and a second side, and wherein the first side and the second side provide a track for sliding the catheter into position in the aperture.

In another embodiment, an absorbent article for use with a medical device affixed to an individual is provided, the absorbent article comprising: a top sheet and a back sheet; an aperture for receiving the medical device, wherein the aperture traverses the top sheet and the back sheet, and wherein the aperture is proximate to a site where the device is affixed to the individual; a reclosable opening that traverses the top sheet and the back sheet, wherein the reclosable opening extends from the aperture to an edge of the absorbent article; and a fastener for closing the reclosable opening.

These and other objects, features, embodiments, and advantages will become apparent to those of ordinary skill in the art from a reading of the following detailed description and the appended claims.

### BRIEF DESCRIPTION OF DRAWINGS

FIG. 1A shows a front view of an absorbent article designed to accommodate an indwelling catheter, wherein the inner surface of the absorbent article faces the viewer.

FIG. 1B shows a back view of an absorbent article designed to accommodate an indwelling catheter, wherein the outer surface of the absorbent article faces the viewer.

FIG. 2A shows a front view of an absorbent article designed to accommodate an indwelling catheter, wherein the absorbent article further comprises a scrotal catch, and wherein the inner surface of the absorbent article faces the viewer.

FIG. 2B shows a back view of an absorbent article designed to accommodate an indwelling catheter, wherein the absorbent article further comprises a scrotal catch, and wherein the outer surface of the absorbent article faces the viewer.

FIG. 3 shows a front view of an insert designed to accommodate an indwelling catheter, wherein the inner surface of the insert faces the viewer.

FIG. 4 shows a back view of an insert designed to accommodate an indwelling catheter, wherein the outer surface of the insert faces the viewer.

FIG. 5 shows a back view of an insert designed to accommodate an indwelling catheter, wherein the outer surface of the insert faces the viewer.

FIG. 6 shows a back view of an insert designed to accommodate an indwelling catheter, wherein the outer surface of the insert faces the viewer.

FIG. 7 shows an insert designed to accommodate an indwelling catheter, positioned in place on an individual in need thereof.

### DESCRIPTION OF EMBODIMENTS

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

For the purposes of describing and defining the present disclosure it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

The term “absorbent article” as used herein refers to devices which are designed to absorb and contain bodily exudates and, more specifically, refers to devices which are placed within, against, or in proximity to, the body of the wearer to absorb and contain the various exudates, such as urine, feces, or vaginal fluids, discharged from the body. Absorbent articles comprise diapers, training pants, adult incontinence absorbent articles, and the like. As used herein the term “diaper” refers to an absorbent article generally used by infants and incontinent persons that is worn about the lower torso of the wearer. Absorbent articles described herein may be disposable in nature.

The term “disposable” is used herein to describe absorbent articles which are generally not intended to be laundered or otherwise restored or reused as an absorbent article (i.e., they are generally intended to be discarded after a single use).

A “catheter” is a hollow flexible tube for insertion into a body cavity, duct, or vessel to allow passage of fluids (such as urine or blood) or to distend a passageway. In a specific embodiment, the catheter is a urinary catheter for the drainage of urine from the bladder.

Certain embodiments of absorbent articles disclosed herein comprise a top sheet, a back sheet, and an optional absorbent core. The “top sheet” is compliant, soft feeling, and non-irritating to the wearer's skin. Further, the top sheet is liquid pervious, permitting liquids (e.g., urine or other liquid waste) to readily penetrate through its thickness. A suitable top sheet may be manufactured from a wide range of materials, such as porous foams, reticulated foams, apertured plastic films, or woven or nonwoven webs of natural fibers (e.g., wood or cotton fibers), synthetic fibers (e.g., polyester or polypropylene fibers), or a combination of natural and synthetic fibers. In one embodiment, the top sheet is made of a hydrophobic material to isolate the wearer's skin from liquids contained in the absorbent article. In another embodiment, the top sheet is treated on at least one side with a surfactant to allow liquids to readily penetrate through its thickness.

The “back sheet” is impervious to liquids (e.g., urine or liquid waste) and may be manufactured from a thin plastic film, although other flexible liquid impervious materials may also be used. As used herein, the term “flexible” refers to materials which are compliant and will readily conform to the general shape and contours of the human body. The back sheet prevents the exudates absorbed and contained in the absorbent article from wetting articles which contact the absorbent article, such as bed sheets, clothing, and undergarments. The back sheet may thus comprise a woven or nonwoven material, polymeric films such as thermoplastic films of polyethylene or polypropylene, or composite materials such as a film-coated nonwoven material. In one embodiment, the back sheet is a thermoplastic film having a thickness of from about 0.012 mm (0.5 mils) to about 0.051 mm (2.0 mils).

The “absorbent core” refers to the portions (e.g., layers) of an absorbent article which function to acquire, distribute, transfer, store and/or redistribute fluid. The absorbent core may comprise any material or combination of materials which is capable of absorbing and retaining liquids such as urine, feces, and other body exudates. The absorbent core may be manufactured from a wide variety of liquid-absorbent materials commonly used in disposable diapers and other absorbent articles such as comminuted wood pulp, which is often referred to in the absorbent article manufacturing industry as airfelt. Examples of other suitable absorbent materials include creped cellulose wadding, melt-blown polymer fibers or mixtures thereof including coform, chemically modified or cross-linked cellulosic fibers, tissue including tissue wraps and tissue laminates, absorbent foams, absorbent sponges, superabsorbent polymers, absorbent gelling materials, or any equivalent material or combination of materials. The configuration and construction of the absorbent core may also be varied (e.g., the absorbent core may have varying caliper zones, hydrophilic gradients, superabsorbent gradients, or lower average density and/or lower average basis weight acquisition zones; or may comprise one or more layers or structures). The total absorbent capacity of the absorbent core should, however, be compatible with the design loading and the intended use of the absorbent article. Further, the size and the absorbent capacity of the absorbent core may be varied to accommodate wearers ranging from infants to adults.

The term “aperture” refers to a hole or opening in the absorbent article or insert. In the case of an absorbent article, the aperture traverses all the layers of the absorbent article—i.e., the top sheet, back sheet, and absorbent core (if present). Similarly, the aperture traverses all layers of the insert—i.e., the non-flexible cup and any other layer of material affixed thereto, such as any lining material added for comfort of the wearer. The aperture may be located anywhere on the absorbent article or insert such that a catheter can pass through the aperture. In one embodiment, the aperture is disposed proximate to an exit site of a catheter from the body, such that the diameter of the catheter conveniently passes through the aperture. In certain embodiments, the edge of the aperture is comprised of a material that provides structure to the aperture and is sufficient to hold the catheter substantially in place while preventing leakage of exudates through the aperture when a catheter is in place. For example, the aperture edge can be comprised of a variety of materials selected from the group consisting of nonwoven fibers, plastics, film, rubber, latex, foam, and combinations thereof. In one embodiment, the aperture edge is configured to support the catheter and substantially hold the catheter in proper position. In another embodiment, the aperture edge is configured to snugly encircle the diameter of the catheter tubing, to prevent leakage of fluid through the interface between the tubing and the aperture edge.

“Reclosable opening” refers to an opening in the absorbent article or insert that facilitates access to the site of exit of the catheter from the body of the wearer and permits placement of the absorbent article or insert on the wearer. In one embodiment, the reclosable opening comprises an opening that traverses all layers of the absorbent article or insert, extending from the aperture to an edge of the absorbent article or insert. The reclosable opening thus comprises a first side and a second side, which sides are joined together when the opening is in a closed configuration. The reclosable opening is configured such that the first and second sides may be joined together to form a seal which substantially prevents leakage of body exudates (e.g., urine or feces) from the opening, aperture, and absorbent article when the first and second sides are in the closed configuration. In one embodiment, the reclosable opening is configured such that the opening may be opened or reclosed by a caregiver without substantially damaging the integrity of the absorbent article or insert. In another embodiment, the first and second sides of the reclosable opening overlap when the first and second sides are in the closed configuration.

“Fastener” refers to a device for attaching two or more elements or portions of elements together in a manner in which they can be separated and re-attached without substantial degradation of fastener performance or damage to surrounding components of the article which would impair the article's continued use. It will be appreciated that a fastener need not have an infinite life span, but it is sufficient that the components attached in a refastenable or releasably fastenable manner can be separated and re-attached successively several times over the typical use life span of the article. It will also be appreciated that the aggressiveness of actual fastening may be reduced significantly from fastening to refastening in absolute terms, but that such reduction is not “substantial degradation” of fastener performance if the resulting refastened strength is sufficient for purposes of ordinarily using the article and fastener. In one embodiment, the fastener holds the first and second sides of the reclosable opening together in a closed configuration. Suitable fasteners include, for example, tape, adhesive, hook and loop closures, hook and hook closures, and tongue and groove fasteners, although any fastener known in the art is generally acceptable and may be employed. In this way, a caregiver positioning the absorbent article on the wearer can open, close, reopen, and reclose the fastener as needed over the typical lifespan of the absorbent article or insert in order to properly position the indwelling catheter.

In the case of an absorbent article such as a disposable diaper having an absorbent core comprising absorbent particles, each of the first and second sides are configured to prevent leakage of absorbent particles from the interior of the absorbent core. For example, the top sheet and back sheet may be fused at the edge of the first and second sides of the reclosable opening to prevent leakage of absorbent particles through the reclosable opening.

In another embodiment, the sides of the reclosable opening are held in the closed configuration by virtue of a fastener, which is disposed along an edge of the first side, the second side, or both sides of the reclosable opening. The fastener can be any device capable of holding the reclosable opening in a closed configuration in a manner that can be reversibly opened and closed without damaging the integrity of the absorbent article or insert. In one embodiment, the fastener is selected from the group consisting of adhesive, hook and loop closures (e.g., Velcro®), tongue and groove closures, and combinations thereof.

In one embodiment, the sides of the reclosable opening are configured to provide a track for sliding the catheter into position in the aperture. Thus, in one embodiment this track is formed from materials which facilitate positioning the catheter in the aperture of the absorbent article. For example, the first and second sides may provide a track comprised of material selected from the group consisting of nonwoven fiber, plastic, film, rubber, latex, foam, and combinations thereof.

“Scrotal catch” refers to an enclosure present in one or more layers configured to support, protect and/or cover at least a portion of a male's scrotum. The scrotal catch may also or alternatively be configured to provide a barrier to reduce and/or prevent fecal matter from contacting the male's penis and/or lower abdominal area. In certain embodiments, the scrotal catch allows for the separation of the interior surface of the absorbent article into an anterior compartment which supports, protects, and/or covers at least a portion of the scrotum, and a posterior compartment comprising the anus, or site of expulsion of feces. This compartmentalization of the absorbent article reduces and/or prevents the fecal matter from entering the anterior compartment and contacting the penis or lower abdomen of the wearer by retaining the fecal matter in the posterior compartment of the absorbent article. In certain embodiments, the scrotal catch is attached to at least a portion of the top sheet and can be positioned behind the scrotum of the wearer, thus separating the penis and lower abdomen from the site of expulsion of feces. See, for example, U.S. Pat. Nos. 8,142,407 and 8,142,408, issued to Reddy.

“Non-flexible,” as used herein, means a material present in one or more layers, which will not readily conform to the general shape and contours of an object. This term includes, but is not limited to, rigid materials, semi-rigid materials, soft-pliable materials with structural elements to maintain a non-flexible form, and combinations thereof. Various materials may be employed for the scrotal catch. Examples of suitable materials include one or more layers of a nonwoven fabric, a woven fabric, a scrim, film, an open-celled foam or any combinations thereof. In addition, it will be appreciated that the one or more layers of the material may display various properties. For example, one or more layers of the scrotal catch may be breathable, liquid permeable, liquid impermeable, flexible, non-flexible or any combination thereof.

The term “medical device,” as used herein, refers to a medical device affixed to an individual. In one embodiment, the absorbent article described herein is configured to accommodate the medical device and/or associated tubing. For example, in one embodiment an absorbent article is provided that is configured to accommodate a pediatric urine collection bag, an indwelling catheter, an external catheter, an insulin pump, an intravenous line, and the like. In a specific embodiment, the absorbent article is a disposable diaper configured to accommodate the medical device and/or associated tubing. For example, in one embodiment a disposable diaper is provided comprising an aperture and a reclosable opening configured to accommodate a pediatric urine collection bag. In such an embodiment, the size and location of the aperture are positioned such that the pediatric urine collection bag can be drawn through the reclosable opening and aperture, the reclosable opening can be closed, and the infant wearing the collection bag can wear a disposable diaper in place to collect fecal waste while also comfortably wearing the pediatric urine collection bag. The aperture and reclosable opening substantially prevent leakage of body exudates from the diaper while the pediatric urine collection bag is in place on the infant.

These and other features of the instantly disclosed subject matter are further disclosed in the accompanying figures.

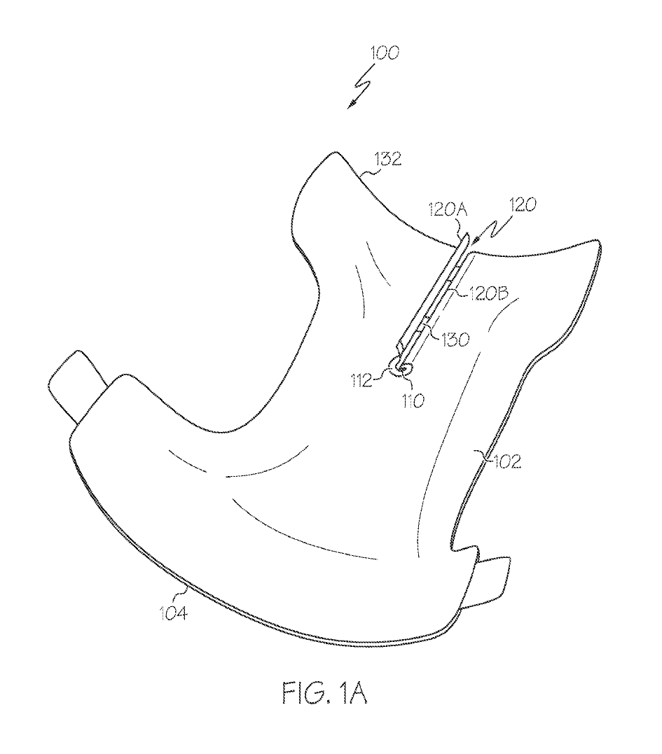

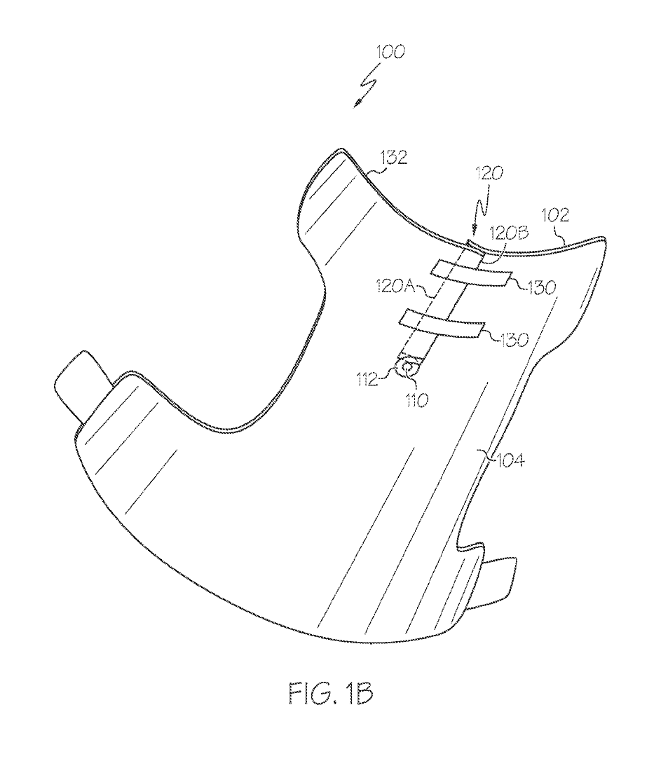

FIG. 1A shows a front view of an absorbent article 100, such as a disposable diaper, wherein the inner surface of the absorbent article 100 is facing the viewer. In the following embodiments, an absorbent article 100 is configured to accommodate a urinary catheter and its associated tubing, however one skilled in the art will understand that other types of catheters and medical devices could also be used with embodiments of the present invention.

The absorbent article 100 comprises a top sheet 102 and a back sheet 104. The absorbent article 100 further comprises an aperture 110, which traverses the top sheet 102 and the back sheet 104. The absorbent article 100 optionally comprises an absorbent core, not shown, disposed between the top sheet 102 and the back sheet 104. When the absorbent core is present, the aperture 110 traverses all layers of the absorbent article 100.

The aperture 110 may be located anywhere on the absorbent article 100 such that a catheter and its associated tubing can pass through the aperture 110. In one embodiment, the aperture 110 is disposed proximate to an exit site of a urinary catheter from the body of the wearer of the absorbent article 100, such that the diameter of the catheter conveniently passes through the aperture 110.

In a specific embodiment, the aperture 110 comprises an aperture edge 112, wherein the aperture edge 112 is comprised of a material that provides structure to the aperture 110 and holds the catheter substantially in place. For example, the aperture edge 112 can be comprised of a variety of materials selected from the group consisting of nonwoven fibers, plastics, film, rubber, latex, foam, and combinations thereof. In one embodiment, the aperture edge 112 is configured to support the catheter and substantially hold the catheter in proper position. In another embodiment, the aperture edge 112 is formed from a material that conforms to the diameter of the catheter and substantially prevents leakage of body exudates, such as urine or feces, from the absorbent article 100 through the aperture 110 when a catheter is in position in the aperture 110.

The absorbent article 100 further comprises a reclosable opening 120, which extends from the aperture 110 to an edge of the absorbent article 100. In FIG. 1, the reclosable opening 120 extends from the aperture 110 to a top front edge 132 of the absorbent article 100, located at the front waistband when the absorbent article 100 is placed on the wearer. The reclosable opening 120 has a first side 120A and a second side 120B. In one embodiment, the sides 120A and 120B of the reclosable opening 120 substantially overlap when in the closed configuration and form a seal, such that the reclosable opening 120 substantially prevents leakage of urine or feces from the absorbent article 100 when the first side 120A and the second side 120B are in a closed configuration. In certain embodiments, the first side 120A and second side 120B are formed from a material that provides structure to the reclosable opening 120, such that the sides 120A and 120B form a track for sliding the catheter into position in the aperture 110.

The absorbent article 100 further comprises a fastener 130, disposed along an edge of the first side 120A, second side 120B, or both sides 120A and 120B of the reclosable opening 120. In the instant embodiment, the fastener 130 comprises adhesive or hook and loop-type closures that traverse the reclosable opening 120 and hold the sides 120A and 120B in the closed configuration. One skilled in the art will appreciate that a variety of fasteners 130 are suitable for use in the embodiments disclosed herein. For example, in another embodiment not shown, the entire length of the sides 120A and 120B may comprise a fastener 130, such as adhesive or mating sides of a hook and loop-type closure or a tongue and groove closure, such that the entire length of the reclosable opening 120 may be secured in the closed configuration.

FIG. 1B shows a back view of an absorbent article 100 designed to accommodate an indwelling catheter, wherein the outer surface of the absorbent article 100 faces the viewer, and wherein like parts are numbered in like manner. FIG. 1B shows the sides 120A and 120B of reclosable opening 120 in the closed configuration, such that the sides 120A and 120B substantially overlap. The fastener 130 traverses the reclosable opening 120 in a perpendicular manner, thus holding the reclosable opening 120 in a closed configuration. In specific embodiments, the fastener 130 comprises adhesive or hook and loop-type closures.

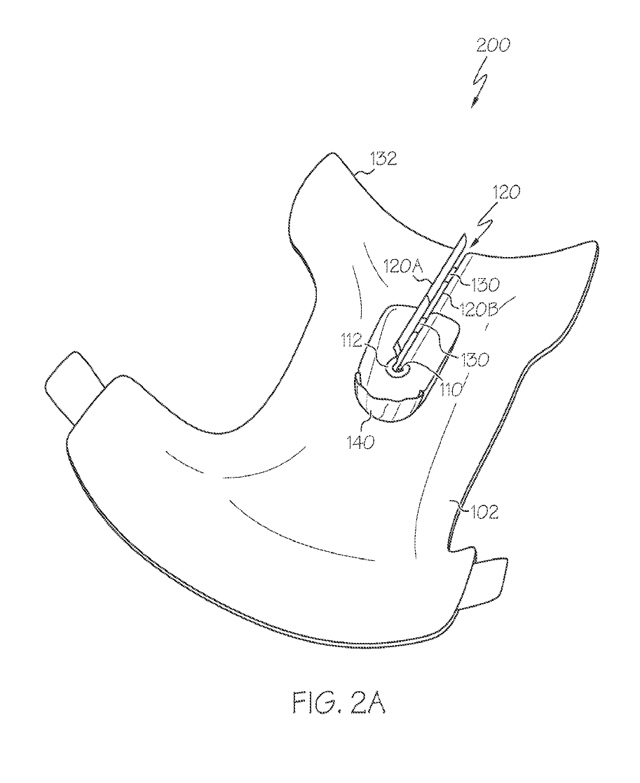

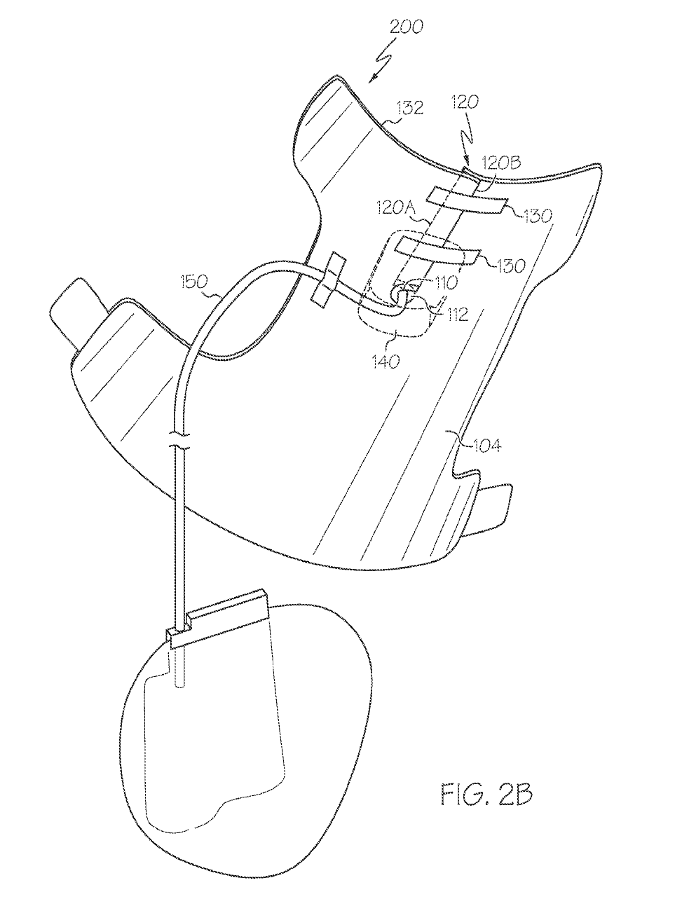

FIG. 2A shows an embodiment of the present invention comprising an absorbent article 200, wherein the inner surface of the absorbent article 200 faces the viewer and wherein like parts are numbered in like manner. Absorbent article 200 comprises a scrotal catch 140 configured to support, protect and/or cover at least a portion of a male wearer's scrotum. The scrotal catch 140 may also or alternatively be configured to provide a barrier to reduce and/or prevent fecal matter from contacting the male's penis and/or lower abdominal area. In certain embodiments, the scrotal catch 140 allows for the separation of the interior surface of the absorbent article 200 into anterior and posterior compartments. This compartmentalization of the absorbent article 200 reduces or prevents the fecal matter from entering the anterior compartment and contacting the penis or lower abdomen of the male wearer by retaining the fecal matter in the posterior compartment of the absorbent article 200.

FIG. 2B shows a back view of an absorbent article 200 designed to accommodate an indwelling catheter, wherein the absorbent article 200 further comprises a scrotal catch 140, and wherein the outer surface of the absorbent article 200 faces the viewer. Like parts are numbered in like manner. FIG. 2B shows catheter tubing 150 extending through the aperture 110, wherein the aperture edge 112 substantially conforms to the diameter of the catheter tubing 150 and prevents leakage of body exudates through the aperture 110 when a catheter is in place. In this view, reclosable opening sides 120A and 120B are in the closed configuration, and secured in place by fastener 130. In one embodiment, the absorbent article may optionally comprise an additional fastener 160 for holding the catheter tubing in place on the exterior of the absorbent article.

FIG. 3 shows a front view of an insert 300 designed to accommodate an indwelling catheter, wherein the inner surface of the insert 300 faces the viewer. The insert 300 is comprised of a non-flexible cup 305 which is does not readily conform to the body surface of the wearer, and a scrotal catch 340, which is flexible and conforms to body contours. The insert 300 provides a physical shield to the lower pelvic area of the wearer and accommodates the exit point of a catheter from the body of the wearer, specifically a urinary catheter. The insert 300 is suitable for use alone or in combination with a garment or absorbent article, such as a diaper or disposable diaper. The insert 300 is also suitable for use with embodiments of the present invention such as, for example, the absorbent article 100 disclosed herein.

The insert 300 has an aperture 310, which may be located anywhere on the insert 300 such that a catheter and/or its associated tubing can pass through the aperture 310. In one embodiment, the aperture 310 is disposed proximate to an exit site of a urinary catheter from the body of the wearer of the insert 300, such that the diameter of the catheter conveniently passes through the aperture 310.

In a specific embodiment, the aperture 310 comprises an aperture edge 312, wherein the aperture edge 312 is comprised of a material that holds the catheter substantially in place. For example, the aperture edge 312 can be comprised of a variety of materials selected from the group consisting of nonwoven fiber, plastics, film, rubber, latex, foam, and combinations thereof. In one embodiment, the aperture edge 312 is configured to support the catheter and substantially hold the catheter in proper position.

The insert 300 comprises a reclosable opening 320, which extends from the aperture 310 to an edge of the insert 300. In the embodiment shown, the reclosable opening 320 extends from the aperture 310 to a top front edge 332 of the insert 300, although one skilled in the art will appreciate that the reclosable opening 320 can extend to any edge of the insert 300. The reclosable opening 320 comprises a first side 320A and a second side 320B. Sides 320A and 320B provide a track for sliding the catheter tubing into place in the aperture 310. Sides 320A and 320B are formed from material that permits positioning of the catheter into place, but substantially closes the gap when the catheter is positioned in the aperture 310. For example, sides 320A and 320B may be formed from nonwoven fiber, plastic, film, rubber, latex, foam, and combinations thereof.

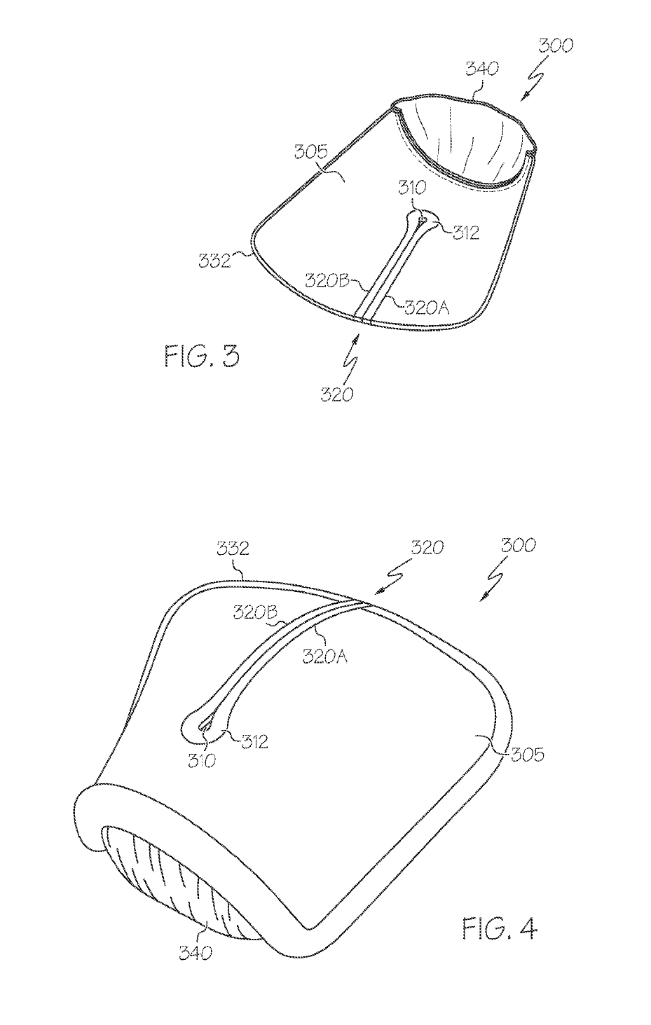

FIG. 4 shows a back view of an insert 300 designed to accommodate an indwelling catheter, wherein the outer surface of the insert 300 faces the viewer and wherein like parts are numbered in like manner. The insert 300 comprises a non-flexible cup 305 and a flexible scrotal catch 340. The insert 300 comprises an aperture 310 comprising an aperture edge 312. The insert 300 further comprises a reclosable opening 320, comprising sides 320A and 320B. FIG. 4 also shows an embodiment of insert 300 comprising a cushioned edge 352 disposed around the perimeter of the non-flexible cup 305. The cushioned edge 352 functions to minimize any irritation to the skin of the wearer caused by contact with the insert 300. The cushioned edge 352 may be formed from a variety of materials including, for example, nonwoven fiber, plastic, film, rubber, latex, foam, and the like.

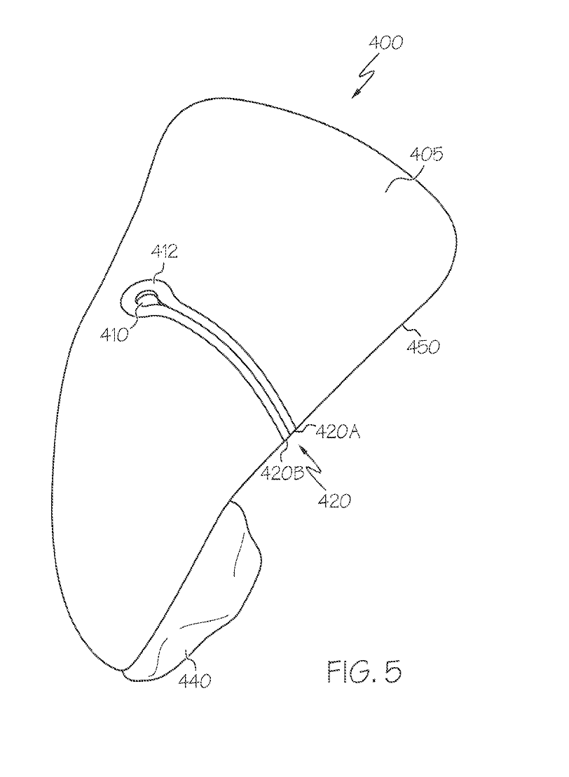

FIG. 5 shows a back view of an insert 400 designed to accommodate an indwelling catheter, wherein the outer surface of the insert 400 faces the viewer. The insert 400 comprises a non-flexible cup 405 and a flexible scrotal catch 440. The insert 400 provides a physical shield to the lower pelvic area of the wearer and accommodates the exit point of a catheter from the body of the wearer, specifically a urinary catheter. The insert 400 is suitable for use alone or in combination with a garment or absorbent article, such as a diaper or disposable diaper. The insert 400 is also suitable for use with embodiments of the present invention such as, for example, the absorbent article 100 disclosed herein.

The insert 400 has an aperture 410, which may be located anywhere on the insert 400 such that a catheter and/or its associated tubing can pass through the aperture 410. In one embodiment, the aperture 410 is disposed proximate to an exit site of a urinary catheter from the body of the wearer of the insert 400, such that the diameter of the catheter conveniently passes through the aperture 410.

In a specific embodiment, the aperture 410 comprises an aperture edge 412, wherein the aperture edge 412 is comprised of a material that holds the catheter substantially in place. For example, the aperture edge 412 can be comprised of a variety of materials selected from the group consisting of nonwoven fiber, plastics, film, rubber, latex, foam, and combinations thereof. In one embodiment, the aperture edge 412 is configured to support the catheter and substantially hold the catheter in proper position.

The insert 400 comprises a reclosable opening 420, which extends from the aperture 410 to an edge of the insert 400. In the embodiment shown, the reclosable opening 420 extends from the aperture 410 to a side edge 450 of the insert 400, although one skilled in the art will appreciate that the reclosable opening 420 can extend to any edge of the insert 400. The reclosable opening 420 comprises a first side 420A and a second side 420B. Sides 420A and 420B provide a track for sliding the catheter tubing into place in the aperture 410. Sides 420A and 420B are formed from material that permits positioning of the catheter into place, but substantially closes the gap when the catheter is positioned in the aperture 410. For example, sides 420A and 420B may be formed from nonwoven fiber, plastic, film, rubber, latex, foam, and combinations thereof.

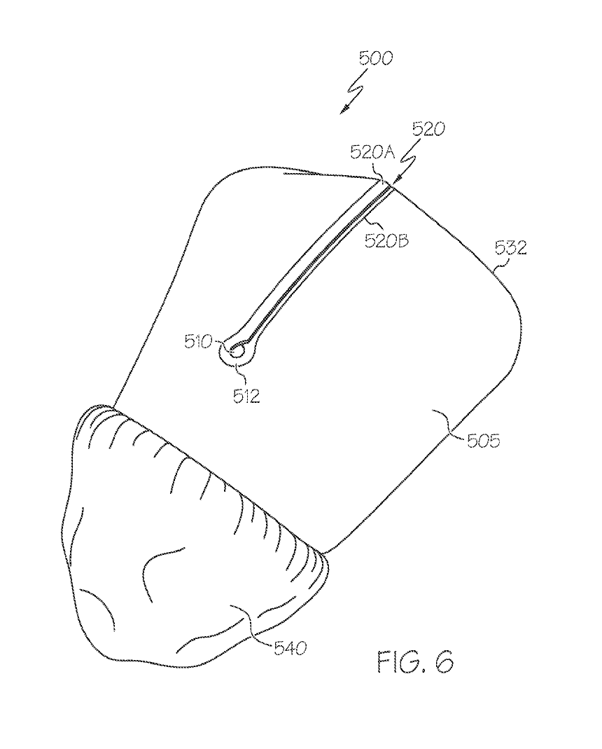

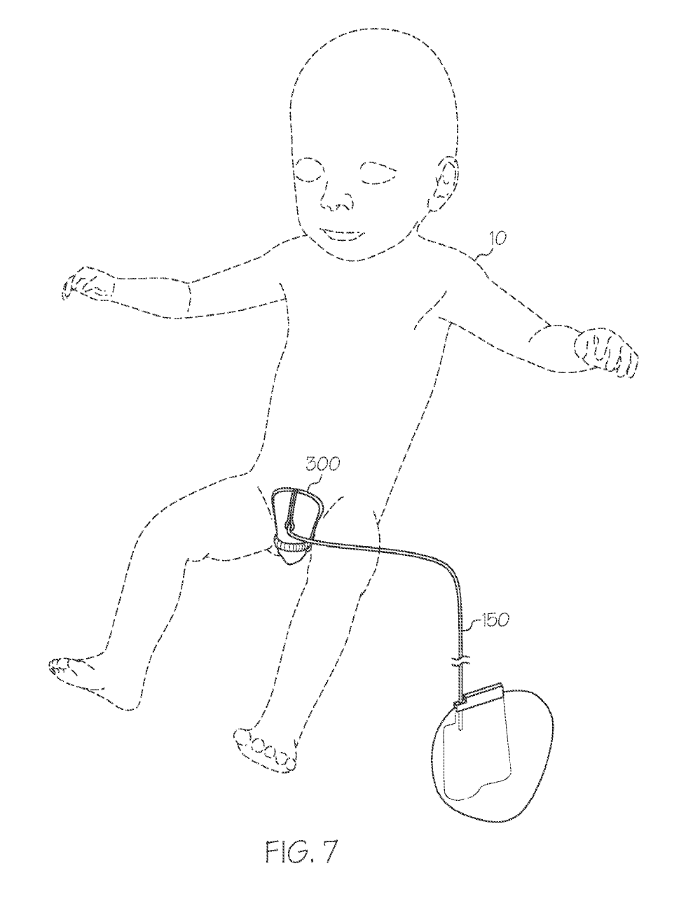

FIG. 6 shows a back view of an insert 500 designed to accommodate an indwelling catheter, wherein the outer surface of the insert 500 faces the viewer. Insert 500 comprises a non-flexible cup 505, which does not conform to the body surface of the wearer and a flexible scrotal catch 540, which is flexible and conforms to the body of the wearer. The insert 500 provides a physical shield to the lower pelvic area of the wearer and accommodates the exit point of a catheter from the body of the wearer, specifically a urinary catheter. The insert 500 is suitable for use alone or in combination with a garment or absorbent article, such as a diaper or disposable diaper. The insert 500 is also suitable for use with embodiments of the present invention such as, for example, the absorbent article 100 disclosed herein.

The insert 500 has an aperture 510, which may be located anywhere on the insert 500 such that a catheter and/or its associated tubing can pass through the aperture 510. In one embodiment, the aperture 510 is disposed proximate to an exit site of a urinary catheter from the body of the wearer of the insert 500, such that the diameter of the catheter conveniently passes through the aperture 510.

In a specific embodiment, the aperture 510 comprises an aperture edge 512, wherein the aperture edge 512 is comprised of a material that holds the catheter substantially in place. For example, the aperture edge 512 can be comprised of a variety of materials selected from the group consisting of nonwoven fiber, plastics, film, rubber, latex, foam, and combinations thereof. In one embodiment, the aperture edge 512 is configured to support the catheter and substantially hold the catheter in proper position.

The insert 500 comprises a reclosable opening 520, which extends from the aperture 510 to an edge of the insert 500. In the embodiment shown, the reclosable opening 520 extends from the aperture 510 to a top front edge 532 of the insert 500, although one skilled in the art will appreciate that the reclosable opening 520 can extend to any edge of the insert 500. The reclosable opening 520 comprises a first side 520A and a second side 520B. Sides 520A and 350B provide a track for sliding the catheter tubing into place in the aperture 510. FIG. 6 shows an embodiment wherein sides 520A and 520B substantially overlap. Sides 520A and 520B are formed from material that permits positioning of the catheter into place, but substantially closes the gap when the catheter is positioned in the aperture 510. For example, sides 520A and 520B may be formed from nonwoven fiber, plastic, film, rubber, latex, foam, and combinations thereof.

Any of the inserts 300, 400, or 500 may further comprise a fastener located along the edge of the first side, second side, or both the first and the second side of the reclosable opening. In such a case, the fastener is disposed on the insert and traverses the reclosable opening.

FIG. 7 shows an insert 300 positioned for use on an individual 10 with a catheter and catheter tubing 150. While insert 300 is depicted in FIG. 7, it is understood that any of the insert embodiments 300, 400, or 500 would be similarly positioned on the individual 10 for use with a catheter, such as a urinary catheter.

All documents cited are incorporated herein by reference in their entirety; the citation of any document is not to be construed as an admission that it is prior art with respect to the present invention.

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

1. An absorbent article for use with an indwelling catheter, comprising:
a top sheet and a back sheet;
an aperture for receiving a catheter, wherein the aperture traverses the top sheet and the back sheet, and wherein the aperture is proximate to an exit site of the catheter from an individual;
a reclosable opening that traverses the top sheet and the back sheet, wherein the reclosable opening comprises a first side and a second side and wherein the reclosable opening extends from the aperture to an edge of the absorbent article;
a fastener for closing the reclosable opening; and
an absorbent core disposed between the top sheet and the back sheet, wherein the aperture and the reclosable opening each traverse the top sheet, the absorbent core, and the back sheet.

2. The absorbent article of claim 1, wherein the first side and the second side of the reclosable opening form a seal such that the reclosable opening substantially prevents leakage of urine or feces from the absorbent article when the first side and the second side are in a closed configuration.

3. The absorbent article of claim 1, wherein the first side and the second side of the reclosable opening substantially overlap when the absorbent article is applied to an individual.

4. The absorbent article of claim 3, wherein a fastener is located along an edge of the first side, the second side, or both the first side and the second side of the reclosable opening.

5. The absorbent article of claim 4, wherein the fastener is selected from the group consisting of adhesive, hook and loop closures, tongue and groove closures, and combinations thereof.

6. The absorbent article of claim 1, wherein the first side and the second side of the reclosable opening provide a track for sliding the catheter into position in the aperture.

7. The absorbent article of claim 6, wherein the track comprises a material selected from the group consisting of nonwoven fiber, plastic, film, rubber, latex, foam, and combinations thereof.

8. The absorbent article of claim 4, wherein the fastener is disposed on the top sheet or the back sheet and traverses the reclosable opening.

9. The absorbent article of claim 1, wherein the aperture comprises an edge, and wherein the edge of the aperture comprises a material selected from the group consisting of nonwoven fiber, plastic, film, rubber, latex, foam, and combinations thereof.

10. The absorbent article of claim 1, wherein the opening extends from the aperture to a top edge of the absorbent article.

11. The absorbent article of claim 1, wherein the absorbent core comprises absorbent particles and wherein the first side and the second side of the opening are configured to prevent leakage of absorbent particles through the reclosable opening.

12. The absorbent article of claim 11, wherein the top sheet and the back sheet are fused at the edge of each of the first side and the second side of the reclosable opening to prevent leakage of absorbent particles.

13. The absorbent article of claim 1, further comprising a scrotal catch.

14. An insert comprising a non-flexible cup, wherein the non-flexible cup comprises:
an aperture for receiving a catheter, wherein the aperture is proximate to an exit site of the catheter from an individual; and
a reclosable opening that extends from the aperture to an edge of the non-flexible cup, wherein the relosable opening comprises a first side and a second side, and wherein the first side and the second side provide a track for sliding the catheter into position in the aperture.

15. The insert of claim 14, wherein the reclosable opening extends from the aperture to a top edge of the non-flexible cup, or wherein the reclosable opening extends from the aperture to a side edge of the non-flexible cup.

16. The insert of claim 14, wherein the track comprises a material selected from the group consisting of nonwoven fiber, plastic, film, rubber, latex, foam, and combinations thereof.

17. The insert of claim 14, wherein the reclosable opening comprises a fastener located along an edge of the first side, the second side, or both the first side and the second side of the reclosable opening, wherein the fastener traverses the reclosable opening.

18. The insert of claim 14, further comprising a scrotal catch.

19. An absorbent article for use with a medical device affixed to an individual, comprising:
a top sheet and a back sheet;
an aperture for receiving the medical device, wherein the aperture traverses the top sheet and the back sheet, and wherein the aperture is proximate to a site where the device is affixed to the individual;
a reclosable opening that traverses the top sheet and the back sheet, wherein the reclosable opening extends from the aperture to an edge of the absorbent article; and
a fastener for closing the reclosable opening.

20. The absorbent article of claim 19, wherein the medical device comprises a pediatric urine collection bag, an indwelling catheter, an external catheter, an insulin pump, or an intravenous line.
